(19) United States
(12) Patent Application Publication
(10) Pub. No.: US 2025/0217986 A1
(43) Pub. Date: Jul. 3, 2025

(54) INFORMATION PROCESSING SYSTEM, INFORMATION PROCESSING METHOD, AND PROGRAM

(71) Applicant: SONY GROUP CORPORATION, TOKYO (JP)

(72) Inventors: NANA KAWAMURA, TOKYO (JP); HIROYASU BABA, TOKYO (JP); TAKANORI FUKAZAWA, TOKYO (JP); MINORI MORITA, TOKYO (JP)

(21) Appl. No.: 18/849,484

(22) PCT Filed: Mar. 14, 2023
(86) PCT No.: PCT/JP2023/009878

§ 371 Date: Sep. 21, 2024

(30) Foreign Application Priority Data
Mar. 31, 2022 (JP) 2022-058221

(51) Int. Cl. G06T 7/00; G06T 7/215; G06T 7/246

(52) U.S. Cl. CPC G06T 7/0016; G06T 7/215; G06T 7/251; G06T 2207/20104; G06T 2207/30061; G06T 2207/30076; G06T 2207/30196

(57) ABSTRACT

The present technology relates to an information processing system, an information processing method, and a program that enable detection of various forms of respiratory abnormalities from a video (image) of a target person.

Positions of joint points of a target person are estimated from an image of the target person captured for each frame, a relative positional relationship between a position of a body region of the target person in the image of a first frame designated by a user and the joint points estimated for the image of the first frame is calculated, the position of the body region of the target person in the image of an arbitrary frame different from the first frame is determined on the basis of the positions of the joint points estimated for the image of the arbitrary frame and the relative positional relationship, and a three-dimensional position change of the body region of the target person is detected on the basis of the position of the body region of the target person in the image of the arbitrary frame.

### TECHNICAL FIELD

The present technology relates to an information processing system, an information processing method, and a program, and more particularly, to an information processing system, an information processing method, and a program capable of detecting various forms of abnormalities in respiration from a video (image) of a target person.

### BACKGROUND ART

Patent Document 1 discloses a technique of acquiring a distance image indicating a distance to a target person, estimating a respiration area of the target person on the basis of the distance image, and detecting respiration of the target person on the basis of a temporal change in a distance from a plane including the respiration area to a three-dimensional position of the target person.

### CITATION LIST

### Patent Document

Patent Document 1: Japanese Patent Application Laid-Open No. 2018-187090

### SUMMARY OF THE INVENTION

### Problems to be Solved by the Invention

There is a demand for a method for detecting an abnormality in respiration of a patient in an ICU or a hospital ward. Abnormality of respiration is not limited to a case where the respiratory rate measured as a vital sign is abnormal, but there is also a case where the way of respiration itself is abnormal, such as a case where one lung is not functioning or a case where only the stomach bulges without the chest bulging due to pneumothorax or the like. Abnormality in the manner of respiration itself is difficult to detect only by vital signs, and can be confirmed only by visual observation by a doctor or a nurse at present.

The present technology has been made in view of such a situation, and enables detection of various forms of respiratory abnormalities from a video (image) of a target person.

### Solutions to Problems

An information processing system or a program according to the present technology is an information processing system including: an estimation unit that estimates positions of joint points of a target person from an image of the target person captured for each frame; a calculation unit that calculates a relative positional relationship between a position of a body region of the target person in the image of a first frame designated by a user and the joint points estimated by the estimation unit for the image of the first frame; a decision unit that determines the position of the body region of the target person in the image of an arbitrary frame different from the first frame on the basis of the positions of the joint points estimated by the estimation unit for the image of the arbitrary frame and the relative positional relationship; and a detection unit that detects a three-dimensional position change of the body region of the target person on the basis of the position of the body region of the target person in the image of the arbitrary frame, or a program for causing a computer to function as such an information processing system.

A data processing method of the present technology is an information processing method in which an information processing system includes an estimation unit, a calculation unit, a decision unit, and a detection unit, in which the estimation unit estimates positions of joint points of a target person from an image of the target person captured for each frame, the calculation unit calculates a relative positional relationship between a position of a body region of the target person in the image of a first frame designated by a user and the joint points estimated by the estimation unit for the image of the first frame, the decision unit determines the position of the body region of the target person in the image of an arbitrary frame different from the first frame on the basis of the positions of the joint points estimated by the estimation unit for the image of the arbitrary frame and the relative positional relationship, and the detection unit detects a three-dimensional position change of the body region of the target person on the basis of the position of the body region of the target person in the image of the arbitrary frame.

In the information processing system, the information processing method, and the program of the present technology, positions of joint points of a target person are estimated from an image of the target person captured for each frame, a relative positional relationship between a position of a body region of the target person in the image of a first frame designated by a user and the joint points estimated for the image of the first frame is calculated, the position of the body region of the target person in the image of an arbitrary frame different from the first frame is determined on the basis of the positions of the joint points estimated for the image of the arbitrary frame and the relative positional relationship, and a three-dimensional position change of the body region of the target person is detected on the basis of the position of the body region of the target person in the image of the arbitrary frame.

### BRIEF DESCRIPTION OF DRAWINGS

FIG. 1 is a block diagram illustrating an information processing system according to an embodiment to which the present technology is applied.

FIG. 2 is a block diagram illustrating a functional configuration of an information processing apparatus.

FIG. 3 is a diagram for explaining a specific determination example in a case where abnormal respiration due to paradoxical respiration is detected by an abnormal respiration determination unit.

FIG. 4 is a diagram for explaining a specific determination example in a case where abnormal respiration due to paradoxical respiration is detected by an abnormal respiration determination unit.

FIG. 5 is a diagram illustrating a candidate region of a body region designation position automatically set with respect to a patient image.

FIG. 6 is a diagram for explaining calculation of relative position information in a relative position calculation unit.

FIG. 7 is a diagram for explaining calculation of relative position information in a relative position calculation unit.

FIG. 8 is a diagram for explaining processing when a body region position decision unit determines a body region setting position.

FIG. 9 is a flowchart for explaining a specific example of a procedure of designating a body region designation position.

FIG. 10 is a diagram illustrating a screen example displayed on the display unit when a body region designation position is designated.

FIG. 11 is a diagram illustrating a screen example displayed on the display unit when a body region designation position is designated.

FIG. 12 is a diagram illustrating a screen example displayed on the display unit when a body region designation position is designated.

FIG. 13 is a diagram illustrating a screen example displayed on the display unit when a body region designation position is designated.

FIG. 14 is a diagram illustrating a screen example displayed on the display unit when a body region designation position is designated.

FIG. 15 is a diagram illustrating a screen example displayed on the display unit when a body region designation position is designated.

FIG. 16 is a block diagram illustrating a configuration example of an embodiment of a computer to which the present technology is applied.

### MODE FOR CARRYING OUT THE INVENTION

Hereinafter, an embodiment of the present technology will be described with reference to the drawings.

### Information Processing System According to Present Embodiment

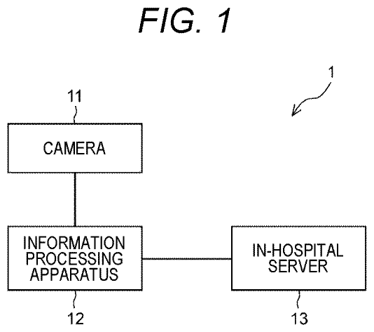

FIG. 1 is a block diagram illustrating an information processing system according to an embodiment to which the present technology is applied. In FIG. 1, the present information processing system 1 includes a camera 11, an information processing apparatus 12, and an in-hospital server 13. The camera 11 is installed in, for example, an intensive care unit (ICU) or a hospital ward, and captures a target person (patient). The camera 11 is, for example, an RGB-D camera, and acquires a color image of a subject and a distance to the subject (distance information (depth information)) as image information. The image information acquired by the camera 11 is supplied to the information processing apparatus 12. Note that the camera 11 may acquire not a color image but a grayscale image, and the color image or the grayscale image acquired by the camera 11 is simply referred to as an image. In particular, since the camera 11 is used for the purpose of acquiring an image of a target person (patient), the image acquired by the camera 11 is referred to as a patient image. Furthermore, the camera 11 acquires a patient image as a moving image by acquiring a patient image at a predetermined cycle (or continuously at predetermined time intervals). A patient image as a moving image is obtained by connecting patient images as still images with different imaging times in chronological order, and each of the patient images as still images is referred to as a frame. The distance information in the image information acquired by the camera 11 is included in each frame. However, the camera 11 may be a camera that does not acquire distance information.

The information processing apparatus 12 is, for example, a general computer connected to a communication network (in-hospital network) in a hospital, and executes a respiratory monitoring process by executing a program included in installed software. Note that the information processing apparatus 12 may be incorporated in any device connected to the in-hospital network. The information processing apparatus 12 monitors the respiration of the target person on the basis of the image information supplied from the camera 11, and supplies the monitoring result information and the image information from the camera 11 to the in-hospital server 13 via the communication network.

The in-hospital server 13 is connected to the in-hospital network and stores monitoring result information and image information from the information processing apparatus 12. The information stored in the in-hospital server 13 can be referred to from a terminal apparatus connected to the in-hospital network.

### <Information Processing Apparatus 12>

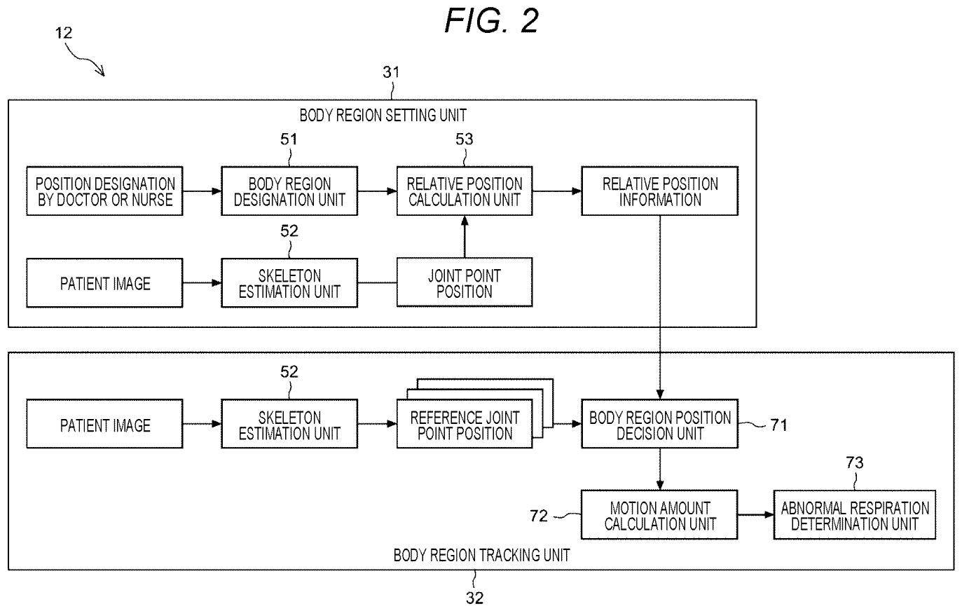

FIG. 2 is a block diagram illustrating a functional configuration of the information processing apparatus 12. The information processing apparatus 12 includes a body region setting unit 31 and a body region tracking unit 32.

The body region setting unit 31 performs body region setting processing of setting a body region to be focused (observed) in order to detect an abnormality of respiration on the patient image acquired by the camera 11.

The body region tracking unit 32 tracks the body region set by the body region setting unit 31 with respect to the patient image acquired by the camera 11, and detects an abnormality in respiration or the like.

The body region setting unit 31 includes a body region designation unit 51, a skeleton estimation unit 52, and a relative position calculation unit 53.

The body region designation unit 51 is an input unit that designates the position of the body region of interest (observation) by the user such as a doctor or a nurse on the screen of the display (display unit) on which the patient image acquired by the camera 11 is displayed using an input apparatus. The input apparatus may be a pointing apparatus such as a mouse that operates a pointer displayed on the screen of the display, or may be a touch panel installed on the screen of the display, and is not limited to a specific type of apparatus. The position (hereinafter, also referred to as a body region designation position) of the body region on the screen (patient image) designated by the body region designation unit 51 is supplied to the relative position calculation unit 53. Specifically, the body region designation position is represented by two-dimensional coordinates (XY coordinates) on the patient image. Note that it is assumed that the front side of the patient in the supine position is captured in the patient image acquired by the camera 11 in principle. In addition, the body region designation position can be designated in both a case where the body region is designated by a point and a case where the body region is designated by a region, and can be designated in both a case where one body region is designated and a case where a plurality of body regions is designated. Details will be described later.

On the basis of the patient image acquired by the camera 11, the skeleton estimation unit 52 performs skeleton estimation in the patient image of the latest frame (current frame) every time a patient image of a new frame is supplied from the camera 11. Skeleton estimation is processing of estimating positions of joint points of a patient on a patient image by inputting the patient image to an inference model (posture estimation model) using a machine learning technology. As the inference model, a posture estimation model generated by a deep learning method such as Pose Proposal Network, Cascaded Pyramid Network (CPN), or GW-Pose is known. The posture inference model may be a model that performs either two-dimensional posture estimation for estimating the positions (two-dimensional positions) of the joint points on the image or three-dimensional posture estimation for estimating the positions (three-dimensional positions) of the joint points in a three-dimensional space. In the present embodiment, since the skeleton estimation unit 52 estimates the two-dimensional position of the joint points on the patient image, a posture inference model that performs two-dimensional posture estimation may be used, or the two-dimensional position of the joint points on the patient image may be calculated from the estimated three-dimensional position of the joint points by using a posture inference model that performs three-dimensional posture estimation. The estimated positions of the joint points (hereinafter, also referred to as a position of a virtual joint point or a virtual joint point position) include, for example, the positions of the shoulders, the elbows, the wrists, the buttocks (waist), the knees, the ankles, the eyes, and the ears existing on the left and right of the human body, and the positions of the neck and the nose existing at the center of the human body. However, the estimated virtual joint point position is not limited thereto. The skeleton estimation unit 52 performs skeleton estimation every time a patient image of a new frame is supplied from the camera 11, and supplies a virtual joint point position of the patient in a patient image of the latest frame (current frame) to the relative position calculation unit 53.

The relative position calculation unit 53 calculates a relative position with respect to a virtual joint point around the body region designation position on the basis of the body region designation position on the patient image from the body region designation unit 51 and the virtual joint point position estimated from the posture image of the frame when the body region designation position is designated, and supplies the relative position to the body region position decision unit 71 of the body region tracking unit 32 as relative position information. Here, the virtual joint points around the body region designation position are at least two or more virtual joint points existing at a close distance to the body region designation position. The relative position calculation unit 53 determines, on the basis of the body region designation positions, joint points (hereinafter, also referred to as a joint point for reference or a reference joint point) in a case where the body region designation positions are represented by relative positions, and calculates, as relative position information, relative positions of the body region designation positions with respect to positions (hereinafter, also referred to as a reference joint point position) of these reference joint points. Note that the relative position information is used as information for specifying the body region of the patient designated by the body region designation unit 51 regardless of the displacement of the patient in the patient image of the new frame sequentially supplied from the camera 11. Therefore, the relative position information indicating the relative position between the body region designation position and the reference joint point position is information for setting the position of the body region of interest of the patient in the patient image of an arbitrary frame, and the position of the body region set on the basis of the relative position information in the patient image of an arbitrary frame is also referred to as a body region setting position, and the relative position information is also referred to as relative position information of the body region setting position. In addition, in the present embodiment, it is assumed that the virtual joint point serving as the reference joint point with respect to the body region designation position is determined in advance with respect to the position of the body region in which the body region designation position is designated.

The body region tracking unit 32 includes a skeleton estimation unit 52, a body region position decision unit 71, a motion amount calculation unit 72 (motion amount detection unit), and an abnormal respiration determination unit 73.

The skeleton estimation unit 52 is a processing unit same as or equivalent to the skeleton estimation unit 52 in the body region setting unit 31, performs skeleton estimation on the basis of the patient image acquired by the camera 11, and estimates a virtual joint point position in the patient image of the latest frame (current frame) every time a patient image of a new frame is supplied from the camera 11. The skeleton estimation unit 52 sets the position of the virtual joint point corresponding to the reference joint point determined by the relative position calculation unit 53 among the virtual joint point positions estimated in the patient image of the current frame as the reference joint point position in the patient image of the current frame, and supplies the position to the body region position decision unit 71 every time a patient image of a new frame is supplied from the camera 11.

Every time a reference joint point position in a patient image of a new frame is supplied from the skeleton estimation unit 52, the body region position decision unit 71 determines the body region setting position in the patient image of the current frame corresponding to the body region designation position designated by the body region designation unit 51 on the basis of the reference joint point position in the patient image of the current frame and the relative position information of the body region setting position from the relative position calculation unit 53. Every time a reference joint point position in a patient image of a new frame is supplied from the skeleton estimation unit 52, the body region position decision unit 71 supplies the determined body region setting position in the patient image of the current frame to the motion amount calculation unit 72. Note that the body region position decision unit 71 may acquire not only information on the reference joint points but also information on virtual joint points other than the reference joint points from the skeleton estimation unit 52, and determine the posture of the patient from the positions of the virtual joint points of both shoulders and the presence or absence of detection of the virtual joint points of the eyes and the nose. As a result, the process of determining the abnormality of the respiration may be performed only in a case where the patient is in the supine position, and the process of determining the abnormality of the respiration may be interrupted in a case where the patient is in the prone position or the lateral decubitus position.

Every time the body region setting position in the patient image of the new frame is supplied from the body region position decision unit 71, the motion amount calculation unit 72 calculates the motion amount of the body region setting position of the current frame on the basis of the body region setting position of the current frame and the patient image of the current frame. The motion amount is, for example, a change amount (referred to as a depth change amount) of the distance (depth value) at the body region setting position indicated by the distance information (depth information) added to the patient image of the current frame with respect to the reference value. The reference value may be a depth value at the body region setting position indicated by the distance information in the patient image of the initial frame, may be an arbitrary value, or may be a temporal average value of the depth values at the body region setting position. Furthermore, the motion amount is a value indicating the magnitude of the motion (three-dimensional position change) of the body region setting position, and is not limited to the depth change amount of the body region setting position, but in the present embodiment, the motion amount is assumed to be the depth change amount. In addition, in a case where the position of the body region of interest is designated by a point as the body region designation position, the body region setting position is also set as the position of the point. In a case where the position of the body region of interest is designated as the body region designation position, the body region setting position is also set as the position of the region. In a case where the body region setting position is set as the position of the point, the motion amount of the body region setting position represents the motion amount at the set point. In a case where the body region setting position is set as the position of the region, the motion amount of the body region setting position may be, for example, an average value, a median value, a maximum value, or a minimum value of the motion amounts at a plurality of points in the set region, and the user may select one of the average value, the median value, the maximum value, or the minimum value as the motion amount of the body region setting position. Every time the body region setting position in the patient image of the new frame is supplied, the motion amount calculation unit 72 supplies the motion amount (depth change amount) calculated for the body region setting position in the patient image of the current frame to the abnormal respiration determination unit 73.

The abnormal respiration determination unit 73 determines whether or not the respiration of the patient is abnormal on the basis of the motion amount at the body region setting position from the motion amount calculation unit 72. For example, the abnormal respiration determination unit 73 tracks the motion of the body region setting position on the basis of the motion amount (depth change amount) of the body region setting position supplied from the motion amount calculation unit 72, and detects a temporal change (depth change) in the motion amount of the body region setting position. As a result, in a case where vigorous movement is detected as compared with the case of normal respiration (normal respiration), for example, in a case where it is detected that the amplitude (variation range) of the depth change exceeds a predetermined threshold value, it is determined that respiration is abnormal (abnormal respiration).

In addition, the abnormal respiration determination unit 73 detects the depth change of the two body region setting positions by the designation of the two body region designation positions by the body region designation unit 51. The abnormal respiration determination unit 73 detects a relative change amount (a temporal change of a difference between the depth change amounts of the two body region setting positions) which is a difference between the depth changes of the two body region setting positions. Note that the difference between the depth changes of the two body region setting positions includes a temporally constant DC component (constant component) due to the difference in the depth value due to the difference in the position of the body region, but the DC component may be removed as the relative change amount. In a case where the movement of the paradoxical respiration is detected as a result of detecting the relative change amount, for example, in a case where it is detected that the variation range of the relative change amount exceeds a predetermined threshold value, the abnormal respiration determination unit 73 determines that the respiration is abnormal (abnormal respiration). The movement of paradoxical respiration refers to a case where the regions of the left and right lungs (chest) do not move symmetrically (depth change), a case where the chest and the abdomen do not move in synchronization, a case where a part of the thorax moves in an opposite manner to the others, and the like. In a case where the abnormal respiration is detected, the abnormal respiration determination unit 73 notifies the abnormality of the respiration by a warning display on a display, a warning sound by a speaker apparatus (not illustrated), or the like.

### <Abnormal Respiration Determination Example in Abnormal Respiration Determination Unit 73>

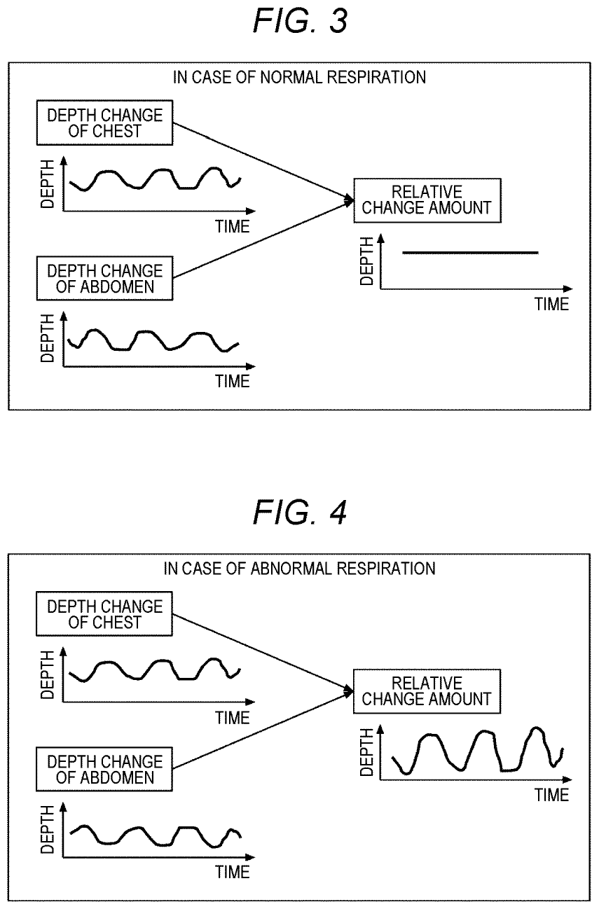

FIGS. 3 and 4 are diagrams for explaining a specific determination example in a case where abnormal respiration due to paradoxical respiration is detected in the abnormal respiration determination unit 73.

On the left side of FIG. 3, the graph representing the depth change of the chest and the graph representing the depth change of the abdomen illustrate temporal changes (depth changes) of the depth values of the chest region and the abdomen region detected as body region setting positions by the abnormal respiration determination unit 73 of the body region tracking unit 32 of FIG. 2 in a case where the chest region and the abdomen region of the patient are designated as the body region designation positions by the body region designation unit 51 of the body region setting unit 31 of FIG. 2.

On the right side of FIG. 3, the graph representing the relative change amount is a graph illustrating a temporal change in a difference between the depth value of the chest region and the depth value of the abdomen region. The graph representing the relative change amount corresponds to a graph representing a difference between the graph representing the depth change of the chest on the left side of FIG. 3 and the graph representing the depth change of the abdomen. FIG. 3 illustrates a case of normal respiration. As in the graph of the relative change amount of FIG. 3, the abnormal respiration determination unit 73 detects the relative change amount from the depth change of two regions of the chest and the abdomen, and determines that the respiration is normal in a case where the variation range of the relative change amount is equal to or less than a predetermined threshold value.

On the left side of FIG. 4, similarly to FIG. 3, the graph representing the depth change of the chest and the graph representing the depth change of the abdomen illustrate temporal changes (depth changes) of the depth values of the chest region and the abdomen region detected as body region setting positions by the abnormal respiration determination unit 73 of the body region tracking unit 32 of FIG. 2 in a case where the chest region and the abdomen region of the patient are designated as the body region designation positions by the body region designation unit 51 of the body region setting unit 31 of FIG. 2.

On the right side of FIG. 4, the graph representing the relative change amount is a graph illustrating a temporal change in a difference between the depth value of the chest region and the depth value of the abdomen region, similarly to FIG. 3. FIG. 4 illustrates a case of abnormal respiration, and the abnormal respiration determination unit 73 detects the relative change amount from the depth change of two regions of the chest and the abdomen as in the graph of the relative change amount of FIG. 4, and determines that the respiration is abnormal in a case where the variation range of the relative change amount exceeds a predetermined threshold value. Since the presence or absence of abnormal respiration of the patient can be constantly monitored by detecting the relative change amount of the plurality of body regions in this manner, it is possible to notice abnormal respiration of the patient at an early stage and to perform appropriate treatment at an early stage. In addition, the data of the presence or absence of abnormal respiration can be reviewed together with the video so that the doctor reviews the patient's vital data. In addition, since the user can designate a plurality of body regions to be monitored, various types of respiratory abnormalities can be detected according to the condition of the patient. In a case where an abnormality in respiration is detected, the doctor or the like is notified of the abnormality, so that the doctor can notice the change in the state of the patient at an early stage, and the severity can be evaluated by looking back on the state of the patient. In addition, since the respiration pattern can be grasped from the video, the respiration pattern can be accurately grasped even when the patient is viewed through the video as in telemedicine.

### <Example of Designating Body Region in Body Region Designation Unit 51>

When the user designates the position of the body region of interest (observation) on the screen of the display (display unit) on which the patient image acquired by the camera 11 is displayed, the body region designation unit 51 of the body region setting unit 31 in FIG. 2 may be able to select from among a plurality of candidate positions (candidate regions) automatically set on the basis of the position of the virtual joint point estimated by the skeleton estimation unit 52.

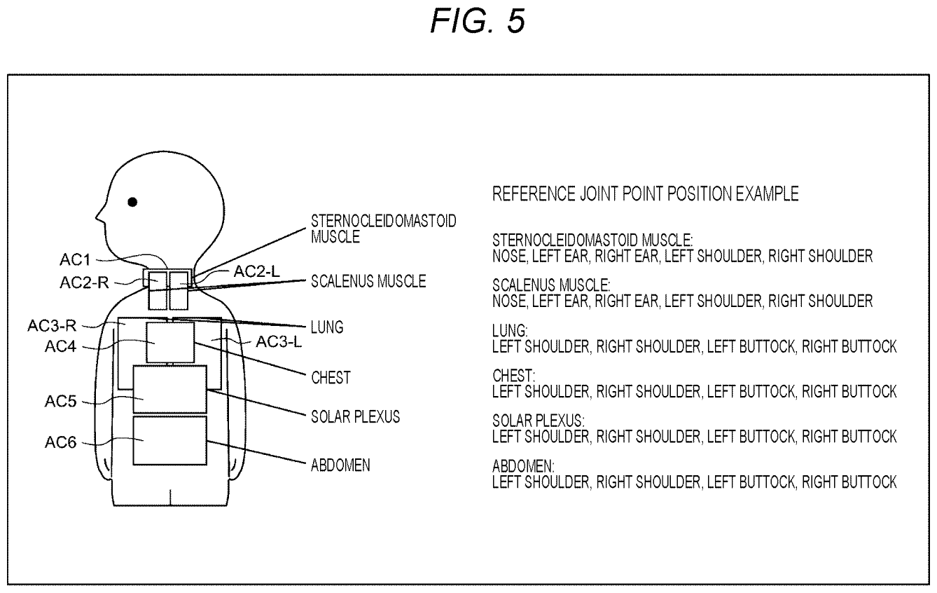

FIG. 5 is a diagram illustrating a candidate region of a body region designation position automatically set for a patient image.

In FIG. 5, candidate regions AC1 to AC6 represent regions of the sternocleidomastoid muscle (AC1), the scalenus muscle (AC2-L and R), the lung (AC3-L and R), the chest (AC4), the solar plexus (AC5), and the abdomen (AC6), which are automatically set as candidate regions of the body region designation position, respectively. The position of the candidate region AC1 of the sternocleidomastoid muscle is set with, for example, the nose, the left ear, the right ear, the left shoulder, and the right shoulder among the virtual joint points estimated by the skeleton estimation unit 52 as reference joint points. For example, the position of each vertex of the candidate region AC1 of the sternocleidomastoid muscle is determined on the basis of a predetermined standard relative positional relationship with respect to the position of each reference joint point.

For example, the nose, the left ear, the right ear, the left shoulder, and the right shoulder among the virtual joint points estimated by the skeleton estimation unit 52 are set as the reference joint points in the left and right candidate regions AC2-L and R of the scalenus muscle, respectively.

For example, the left shoulder, the right shoulder, the left buttock, and the right buttock among the virtual joint points estimated by the skeleton estimation unit 52 are set as the reference joint points in the left and right candidate regions AC3-L and R of the lung.

For example, the left shoulder, the right shoulder, the left buttock, and the right buttock among the virtual joint points estimated by the skeleton estimation unit 52 are set as the reference joint points in the candidate regions AC4, AC5, and AC6 of the chest, the solar plexus, and the abdomen.

For example, by selecting a body region of interest (observation) by the user from the sternocleidomastoid muscle, the scalenus muscle, the lung, the chest, the solar plexus, and the abdomen by the body region designation unit 51, a candidate region corresponding to the selected body region is designated as the body region designation position, or is superimposed and displayed on the patient image displayed on the display (display unit) as the candidate region. The user can also change the shape and position of the body region designated as the body region designation position with respect to the candidate region with reference to the candidate region superimposed and displayed on the patient image.

### <Determination of Body Region Setting Position>

The body region position decision unit 71 of the body region tracking unit 32 in FIG. 2 determines the body region setting position in the patient image of each frame corresponding to the body region designation position designated by the body region designation unit 51 on the basis of the reference joint point position in the patient image of each frame from the skeleton estimation unit 52 and the relative position information of the body region setting position from the relative position calculation unit 53. The relative position calculation unit 53 calculates the relative position information on the basis of the reference joint point position in the patient image of the frame when the position of the body region is designated by the body region designation unit 51 and the position of the body region designated by the user.

### (Calculation of Relative Position Information)

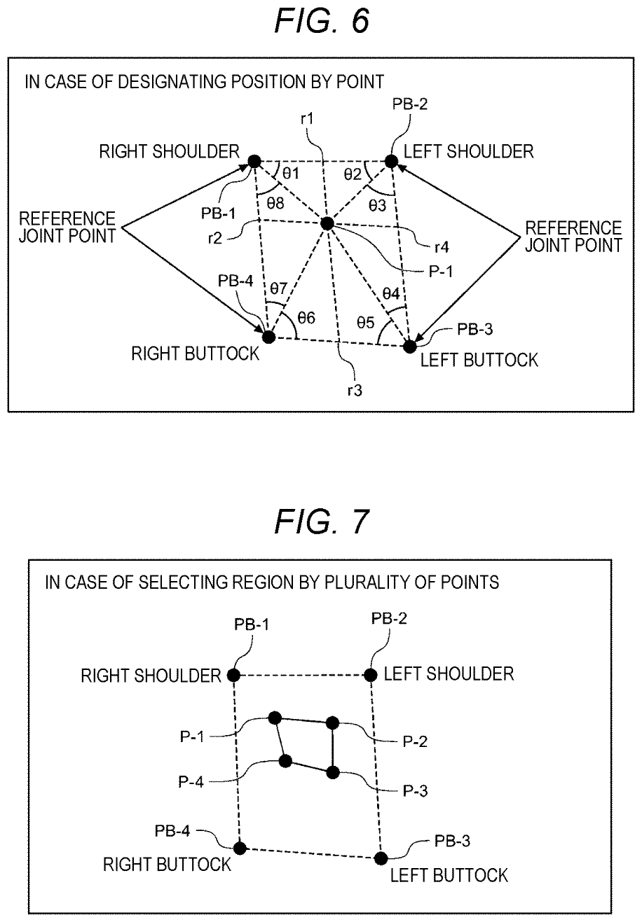

FIG. 6 is a diagram for explaining calculation of relative position information in the relative position calculation unit 53 in a case where the body region designation position is designated by a point. In FIG. 6, a designation point P-1 represents a point designated as a body region designation position in the patient image by the user by the body region designation unit 51. The reference joint points PB-1 to PB-4 represent the positions of the virtual joint points in the patient image in a case where the designation point P-1 is represented by the relative position as the body region designation position, and represent the positions of the joint points of the right shoulder, the left shoulder, the left buttock, and the right buttock estimated by the skeleton estimation unit 52 in FIG. 2, respectively. At this time, the relative position calculation unit 53 calculates an angle θk (angle θk) formed by a line segment (straight line) connecting any one of the reference joint points PB-1 to PB-4 (referred to as a focused joint point PB-m) and a reference joint point other than the focused joint point PB-m (referred to as another reference joint point PB-n) and a line segment (straight line) connecting the focused joint point PB-m and the designation point P-1. In addition, the relative position calculation unit 53 calculates an angle θl (angle θl) formed by a line segment (straight line) connecting the focused joint point PB-m and the other reference joint point PB-n and a line segment (straight line) connecting the other reference joint point PB-n and the designation point P-1. Here, since the position (coordinates) of the designation point P-1 in the patient image can be specified as a relative position with respect to the reference joint points PB-1 to PB-4 if at least one set of angles θk and θl is specified, the relative position calculation unit 53 calculates one or more sets of angles θk and θl as relative position information. In FIG. 6, angles θ1 to θ8 are illustrated, and represent angles θk and θl calculated in a case where the combinations of the focused joint point PB-m and the other reference joint points PB-n (also referred to as combinations of reference joint points) are as follows. The angles θ1 and θ2 represent angles θk and θl calculated by a combination of the reference joint points PB-1 and PB-2. The angles θ3 and θ4 represent angles θk and θl calculated by a combination of the reference joint points PB-2 and PB-3. The angles θ5 and θ6 represent angles θk and θl calculated by a combination of the reference joint points PB-3 and PB-4. The angles θ7 and θ8 represent angles θk and θl calculated by a combination of the reference joint points PB-1 and PB-4. As described above, the relative position calculation unit 53 selects the focused joint point PB-m and the other reference joint point PB-n from the reference joint points PB-1 to PB-4, creates one or more combinations of the reference joint points, and calculates one set of angles θk and θl for each combination. Note that if two or more virtual joint points are set as the reference joint points for one designation point P-1, the relative position calculation unit 53 can create one or more combinations of the reference joint points, and can calculate the angles θk and θl with respect to the combinations as the relative position information of the designation point P-1. In a case where four reference joint points are set as illustrated in FIG. 6, there are a maximum of six combinations of reference joint points. The position of the designation point P-1 can be specified by the position (coordinates) of the reference joint point and a set of angles θk and θl which are relative position information with respect to the combination in any one of the combinations. Therefore, the relative position calculation unit 53 is only required to calculate the angles θk and θl with respect to one or more combinations of reference joint points among all combinations of reference joint points as the relative position information. However, when the angles θk and θl with respect to many combinations of reference joint points are calculated as the relative position information, the reliability of the body region setting position determined by the body region position decision unit 71 on the basis of the relative position information is improved. Therefore, in the present description using FIG. 6, the angles θ1 to θ8 with respect to the combinations of four reference joint points are calculated as the relative position information.

Furthermore, in FIG. 6, the relative position calculation unit 53 sets a line segment (straight line) connecting any one reference joint point (a first focused joint point PB-m1, for example, PB-1) of the reference joint points PB-1 to PB-4 and a reference joint point (another reference joint point PB-n1, for example, PB-2) other than the first focused joint point PB-m1 as a first line segment (straight line), and sets a line segment connecting the reference joint point (a second focused joint point PB-m2, for example, PB-4) other than the first focused joint point PB-m1 and the other reference joint point PB-n1 and the reference joint point (another reference joint point PB-n2, for example, PB-3) other than the second focused joint point PB-m2 as a second line segment (straight line). The relative position calculation unit 53 specifies, as the inner division points ri and rj (for example, r1 and r3), points at which straight lines (referred to as index straight lines) passing through the designation point P-1 and satisfying a predetermined condition (referred to as a linear condition) intersect the first line segment and the second line segment, respectively.

As the linear condition of the index straight line, any of a plurality of forms can be adopted. As a form (first form) of the linear condition, for example, the inclination of the index straight line is an average of the inclination of the line segment (straight line) connecting the first focused joint point PB-m1 and the second focused joint point PB-m2 and the inclination of the line segment (straight line) connecting the reference joint point PB-n1 and the reference joint point PB-n2. As another form (second form) of the linear condition, for example, it is assumed that the index straight line is a straight line passing through inner division points ri and rj (=ri) that internally divide the first line segment and the second line segment at the same inner division ratio. As another form (third form) of the linear condition, it is assumed that the inclination of the index straight line is a predetermined inclination. For example, it is assumed that the index straight line is a straight line in the vertical direction or the horizontal direction on the patient image.

Here, one set of inner division points ri and rj specifies one index straight line passing through the designation point P-1. When at least two index straight lines having different inclinations are specified, the position (coordinates) of the designation point P-1 in the patient image is specified as the position of the intersection of these index straight lines. Therefore, the relative position calculation unit 53 calculates, as the relative position information, information of two or more sets of inner division points ri and rj that specify two or more index straight lines having different inclinations with respect to the reference joint points PB-1 to PB-4. As the information of the inner division points ri and rj, the relative position calculation unit 53 calculates, for example, an inner division ratio at which the inner division points ri and rj respectively internally divide the first line segment and the second line segment. Assuming that the inner division ratios at which the inner division points ri and rj internally divide the line segments are represented by the same reference signs ri and rj as the inner division points, the relative position calculation unit 53 calculates the inner division ratios ri and rj for the first line segment and the second line segment. The inner division ratio ri with respect to the first line segment is a value obtained by dividing the length from the first focused joint point PB-m1 to the inner division point ri on the first line segment by the length of the first line segment, and corresponds to the length from the first focused joint point PB-m1 to the inner division point ri when the length of the first line segment is 1. That is, the inner division ratio ri represents the value of ri in a case where the first line segment is divided by the ratio (ri:1-ri) by the index straight line. Similarly, the inner division ratio rj with respect to the second line segment is a value obtained by dividing the length from the second focused joint point PB-m2 to the inner division point rj on the second line segment by the length of the second line segment, and corresponds to the length from the second focused joint point PB-m2 to the inner division point rj when the length of the second line segment is 1. That is, the inner division ratio rj represents the value of rj in a case where the second line segment is divided by the ratio (rj:1-rj) by the index straight line.

FIG. 6 illustrates inner division points r1 to r4 in a case where the first form of the linear condition of the index straight line is adopted, and represents inner division points ri to rj in which a combination of a first focused joint point PB-m1 and another reference joint point PB-n1 (also referred to as a combination of reference joint points with respect to the first line segment) forming a first line segment and a combination of a second focused joint point PB-m2 and another reference joint point PB-n2 (also referred to as a combination of reference joint points with respect to the second line segment) forming a second line segment are specified in the following cases. The inner division points r1 and r3 are inner division points ri to rj in a case where the reference joint points PB-1 and PB-2 (the reference joint point PB-1 is a first focused joint point PB-m1) are a combination of the reference joint points with respect to the first line segment, and the reference joint points PB-4 and PB-3 (the reference joint point PB-4 is a second focused joint point PB-m2) are a combination of the reference joint points with respect to the second line segment. The inner division points r2 and r4 are inner division points ri to rj in a case where the reference joint points PB-1 and PB-4 (the reference joint point PB-1 is a first focused joint point PB-m1) are a combination of the reference joint points with respect to the first line segment, and the reference joint points PB-2 and PB-3 (the reference joint point PB-2 is a second focused joint point PB-m2) are a combination of the reference joint points with respect to the second line segment.

As the relative position information of the designation point P-1, the relative position calculation unit 53 calculates the inner division ratios r1 and r3 of the inner division points r1 and r3 as a set of inner division ratios ri to rj with respect to one index straight line (first index straight line), and calculates the inner division ratios r2 and r4 of the inner division points r2 and r4 as a set of inner division ratios ri to rj with respect to the other one index straight line (second index straight line).

As described above, the relative position calculation unit 53 selects the first focused joint point PB-m1 and the reference joint point PB-n1, and the second focused joint point PB-m2 and the reference joint point PB-n2 from among the reference joint points PB-1 to PB-4, and creates a combination of the reference joint points with respect to a plurality of (a plurality of sets of) first line segments and second line segments (a plurality of index straight lines having different inclinations). The relative position calculation unit 53 calculates the inner division ratios ri and rj for the combination of the reference joint points with respect to the first line segment and the second line segment of each set. Note that, since it is sufficient that any one of the first line segment and the second line segment of each set is different from the first line segment and the second line segment of the other set, the relative position calculation unit 53 may create one or more line segments larger than the number of sets of the first line segment and the second line segment for calculating the inner division ratios ri and rj. In addition, in the first line segment and the second line segment for the first line segment, if the second focused joint point PB-m2 is different from the first focused joint point PB-m1 and the other reference joint point PB-n1, the other reference joint point PB-n2 for the second focused joint point PB-m2 may be common to the first focused joint point PB-m1 and the other reference joint point PB-n1. Therefore, if three or more reference joint points exist, two or more line segments can be created. For example, in a case where a number of reference joint points a exists, {a·(a −1)/2}line segments can be created at the maximum, and {{a·(a −1)/2}−1}number of first line segments and second line segments (sets) can be created. In a case where there are four reference joint points PB-1 to PB-4 as illustrated in FIG. 6, a maximum of six line segments can be created, and the relative position calculation unit 53 can calculate the inner division ratios ri and rj with respect to five sets of the first line segment and the second line segment (five index straight lines). However, in the present description, as in the example of FIG. 6, it is assumed that the relative position calculation unit 53 calculates, as the relative position information, a set of the inner division ratios r1 and r3 and a set of the inner division ratios r2 and r4 for two sets of the first line segment and the second line segment (two index straight lines).

Note that there may be a case where the index straight line does not have an inner division point that internally divides the first line segment or the second line segment and passes through an outer division point that externally divides the first line segment or the second line segment. In this case, the relative position calculation unit 53 calculates the outer division ratio ri or rj by rephrasing the inner division point described above as the outer division point and rephrasing the inner division ratio ri or rj as the outer division ratio ri or rj. However, since the algorithm in a case where the relative position calculation unit 53 calculates the outer division ratio is similar to that in a case where the inner division ratio is calculated, in the following description, in a case where ri or rj is a value other than the range of 0 to 1, it is assumed that ri or rj is the outer division ratio, and it is assumed that ri and rj (or the ratio value) are the inner division ratios regardless of whether the value ri or rj calculated by the relative position calculation unit 53 is the inner division ratio or the outer division ratio. In addition, in a case where the third form of the linear condition of the index straight line is adopted, the relative position calculation unit 53 calculates the inner division ratios rj of the inner division ratios ri and rj with respect to the first line segment and the second line segment of each set as the same value as the inner division ratio ri.

As described above, the relative position calculation unit 53 calculates the angles θ1 to θ8 and the inner division ratios (ratio values) r1 to r4 of FIG. 6 for the designation point P-1 as the relative position information of the body region setting position, and supplies the angles θ1 to θ8 and the inner division ratios (ratio values) r1 to r4 to the body region position decision unit 71 of FIG. 2. Note that the relative position information also includes information for specifying the types of reference joint points used in calculating each of the angles θ1 to θ8 and the inner division ratios (ratio values) r1 to r4.

FIG. 7 is a diagram for explaining calculation of relative position information in the relative position calculation unit 53 in a case where a body region designation position is designated by a region.

In FIG. 7, in a case where the user designates a region (designation region A-1) as the body region designation position in the patient image by the body region designation unit 51, designation points P-1 to P-4 represent vertices of the designation region A-1. In this case, the user designates the position of the designation region A-1 by designating the position of each of the designation points P-1 to P-4, which are vertices of the designation region A-1, in the patient image. Since the reference joint points PB-1 to PB-4 are the same as those in FIG. 6, the description thereof will be omitted.

At this time, the relative position calculation unit 53 calculates the angles θ1 to θ8 and the inner division ratios (ratio values) r1 to r4 for each of the designation points P-1 to P-4, which are vertices of the designation region A-1, similarly to FIG. 6. Since the calculation procedure is similar to that in FIG. 6, the description thereof is omitted. The relative position calculation unit 53 supplies the angles θ1 to θ8 and the inner division ratios (ratio values) r1 to r4 for each of the designation points P-1 to P-4 to the body region position decision unit 71 in FIG. 2 as relative position information of the body region setting positions.

### (Calculation of Body Region Setting Position)

On the other hand, the body region position decision unit 71 in FIG. 2 determines (calculates) the body region setting position in the patient image of each frame corresponding to the body region designation position designated by the body region designation unit 51 on the basis of the relative position information of the body region setting position from the relative position calculation unit 53 and the reference joint point position in the patient image of each frame supplied from the skeleton estimation unit 52.

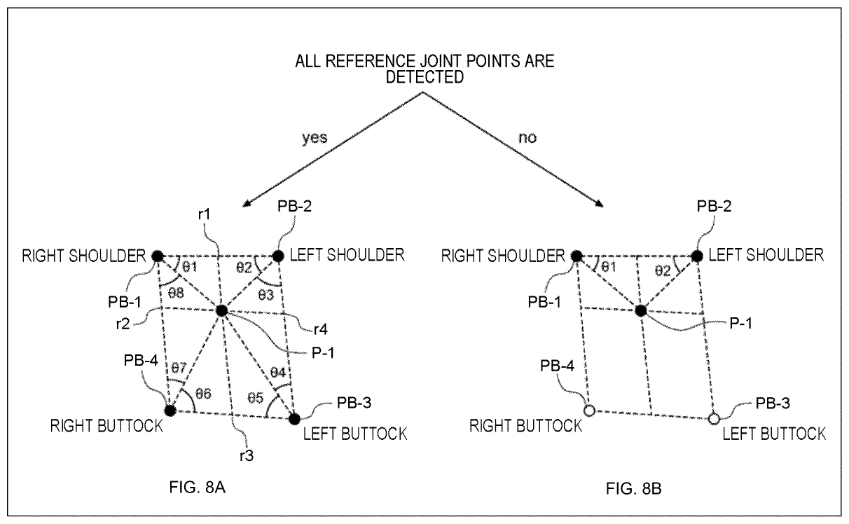

FIG. 8 is a diagram for explaining processing when the body region position decision unit 71 determines the body region setting position with respect to the designation point P-1 in FIG. 6.

In FIG. 8, a setting point P-1 represents a point that the body region position decision unit 71 determines (sets), as the body region setting position in the patient image of a predetermined frame (frame of interest), the designation point P-1 designated as the body region designation position by the user in the patient image by the body region designation unit 51 in FIG. 6. The reference joint points PB-1 to PB-4 represent the positions of the reference joint points in the patient image of the frame of interest corresponding to the reference joint points PB-1 to PB-4 used when the relative position calculation unit 53 calculates the relative position information of the designation point P-1 in FIG. 6, and represent the positions of the joint points of the right shoulder, the left shoulder, the left buttock, and the right buttock estimated with respect to the patient image of the frame of interest by the skeleton estimation unit 52 in FIG. 2, respectively.

In a case where the positions of all the reference joint points used to calculate the correlation position information are estimated and supplied by the skeleton estimation unit 52 with respect to the patient image of the frame of interest, the body region position decision unit 71 calculates the position of the setting point P-1 on the basis of the angles θ1 and θ8 and the inner division ratios (ratio values) r1 to r4 of FIG. 6 with respect to the designation point P-1 supplied as the relative position information from the relative position calculation unit 53 and the positions of the reference joint points PB-1 to PB-4 as illustrated in A of FIG. 8. At this time, the position of the setting point P-1 can be specified from any of the information of the angles θ1 and θ2, the angles θ3 and θ4, the angles θ5 and θ6, the angles θ7 and θ8, and the ratio values r1 to r4, but the body region position decision unit 71 calculates the setting point P-1 as the body region setting position as follows using the weighting coefficient.

The body region position decision unit 71 calculates the positions (XY coordinates) of the setting point P-1 specified from the angles θ1 and θ2, the angles θ3 and θ4, the angles θ5 and θ6, the angles θ7 and θ8, and the inner division ratios (ratio values) r1 to r4 as P-1 (x, y)ₐ₁, P-1 (x, y)ₐ₂, P-1 (x, y)ₐ₃, P-1(x, y)ₐ₄, and P-1(x, y)_{b}. That is, P-1(x, y)ₐ₁ is a setting point specified from the positions (XY coordinates) of the reference joint points PB-1 and PB-2 and the angles θ1 and θ2 in the frame of interest. Specifically, P-1(x, y)ₐ₁ represents a position of an intersection of a straight line passing through the reference joint point PB-1 at an angle θl and a straight line passing through the reference joint point PB-2 at an angle θ2 with respect to a line segment connecting the reference joint points PB-1 and PB-2. P-1(x, y)ₐ₂ is a setting point specified from the positions (XY coordinates) of the reference joint points PB-2 and PB-3 and the angles θ3 and θ4 in the frame of interest. Specifically, P-1(x, y)ₐ₂ represents a position of an intersection of a straight line passing through the reference joint point PB-2 at an angle θ3 and a straight line passing through the reference joint point PB-3 at an angle θ4 with respect to a line segment connecting the reference joint points PB-2 and PB-3. P-1(x, y)ₐ₃ is a setting point specified from the positions (XY coordinates) of the reference joint points PB-3 and PB-4 and the angles θ5 and θ6 in the frame of interest. Specifically, P-1(x, y)ₐ₃ represents a position of an intersection of a straight line passing through the reference joint point PB-3 at an angle θ5 and a straight line passing through the reference joint point PB-4 at an angle θ6 with respect to a line segment connecting the reference joint points PB-3 and PB-4. P-1(x, y)ₐ₄ is a setting point specified from the positions (XY coordinates) of the reference joint points PB-4 and PB-1 and the angles θ7 and θ8 in the frame of interest. Specifically, P-1(x, y)ₐ₄ represents a position of an intersection of a straight line passing through the reference joint point PB-4 at an angle θ7 and a straight line passing through the reference joint point PB-1 at an angle θ8 with respect to a line segment connecting the reference joint points PB-4 and PB-1. P-1(x, y)_{b} is a setting point specified from the positions (XY coordinates) of the reference joint points PB-1 to PB-4 in the frame of interest and the inner division ratios (ratio values) r1 to r4. Specifically, P-1(x, y)_{b} represents a position of an intersection between a line segment (first index straight line) connecting the inner division points r1 and r3 internally dividing the first line segment connecting the reference joint points PB-1 and PB-2 and the second line segment connecting the reference joint points PB-4 and PB-3 at the inner division ratios r1 and r3, respectively, and a line segment (second index straight line) connecting the inner division points r2 and r4 internally dividing the first line segment connecting the reference joint points PB-1 and PB-4 and the second line segment connecting the reference joint points PB-2 and PB-4 at the inner division ratios r2 and r4, respectively.

For these calculated P-1(x, y)ₐ₁, P-1(x, y)ₐ₂, P-1(x, y)ₐ₃, P-1 (x, y)ₐ₄, and P-1 (x, y)_{b}, the body region position decision unit 71 determines P-1(x, y) as the final position (XY coordinates) of the setting point P-1 by the weighted addition sum (the weighted average of each of the X coordinate value and the Y coordinate value) of the following Equation (1) using predetermined weighting coefficients Wₐ₁, Wₐ₂, Wₐ₃, Wₐ₄, and W_{b}. Note that the sum of the weighting coefficients Wₐ₁, Wₐ₂, Wₐ₃, Wₐ₄, and W_{b} is 1.

$$P - 1 ( x , y ) = {W}_{a 1} \cdot P - 1 {( x , y )}_{a 1} + {W}_{a 2} \cdot P - 1 {( x , y )}_{a 2} + {W}_{a 3} \cdot P - 1 {( x , y )}_{a 3} + {W}_{a 4} \cdot P - 1 {( x , y )}_{a 4} + {W}_{b} \cdot P - 1 {( x , y )}_{b} \qquad ( 1 )$$

Note that, regarding the weighting coefficients Wₐ₁, Wₐ₂, Wₐ₃, Wₐ₄, and W_{b}, the weighting coefficient W_{b} for P-1(x, y)_{b} calculated from the inner division ratios (ratio values) r1 to r4 with high reliability of position specification is set to a value larger than the weighting coefficients Wₐ₁, Wₐ₂, Wₐ₃, and Wₐ₄, so that the position of the setting point P-1 with high reliability and high robustness is calculated as the designation region setting position. In addition, the above Equation (1) is a case where the position (XY coordinates) of one setting point P-1(x, y)_{b} is calculated on the basis of the inner division ratios r1 and r3 and the inner division ratios r2 and r4 of two sets with respect to the first line segment and the second line segment (two sets) of two sets (two index straight lines). On the other hand, in a case where the positions (XY coordinates) of two or more setting points are calculated on the basis of the inner division ratios ri and rj of three or more sets with respect to three or more sets of the first line segment and the second line segment (three or more index straight lines), the positions of the plurality of setting points may also be added as a weighted addition sum similarly to the above Equation (1). Furthermore, the body region position decision unit 71 is not limited to the case of determining the final position of the setting point P-1 by the weighted addition sum (weighted average) of the candidate positions of all the setting points P-1 as in the above Equation (1) in a case where there are the positions (candidate positions) of the plurality of setting points P-1 that can be specified on the basis of the relative position information from the relative position calculation unit 53 with respect to one designation point P-1. For example, the body region position decision unit 71 may determine one of the plurality of candidate positions as the final position of the setting point P-1, or may determine the final position of the setting point P-1 by a weighted addition sum of some candidate positions among the plurality of candidate positions.

On the other hand, in a case where the skeleton estimation unit 52 does not estimate the position of any reference joint point among the reference joint points used to calculate the correlation position information with respect to the patient image of the frame of interest and does not supply the position of the reference joint point, the body region position decision unit 71 calculates the position of the setting point P-1 using only the information capable of calculating the position of the setting point P-1 in the relative position information. For example, in a case where the positions of the reference joint points PB-3 and PB-4 cannot be estimated as in B of FIG. 8, the position of the setting point P-1 is calculated using the reference joint points PB-1 and PB-2 and the angles θ1 and θ2 of the relative position information. That is, in Equation (1), assuming that the weighting coefficient Wₐ₁ is 1, and that Wₐ₂, Wₐ₃, Wₐ₄, and W_{b} are 0, P-1(x, y) is calculated as the final position (XY coordinates) of the setting point P-1.

As a result, the body region position decision unit 71 determines the body region setting position corresponding to the body region designation position designated by the body region designation unit 51 in the patient image of the frame of interest. Note that, in a case where the body region designation position is designated by the region (designation region A-1) as illustrated in FIG. 7, the body region position decision unit 71 calculates the positions (XY coordinates) of the setting points P-1 to P-4 in the frame of interest corresponding to the designation points P-1 to P-4, which are vertices of the designation region A-1, on the basis of the relative position information similarly to FIG. 8 in a case where the body region designation position is designated by a point. The body region position decision unit 71 determines a region having the calculated positions of the setting points P-1 to P-4 as vertices as the body region setting positions.

### <Example of Procedure for Designating Body Region Designation Position>

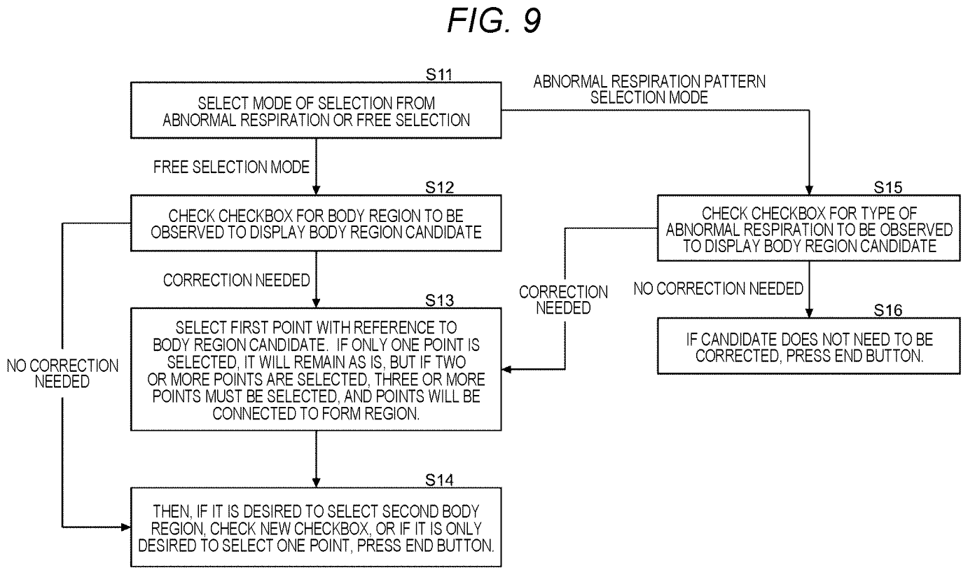

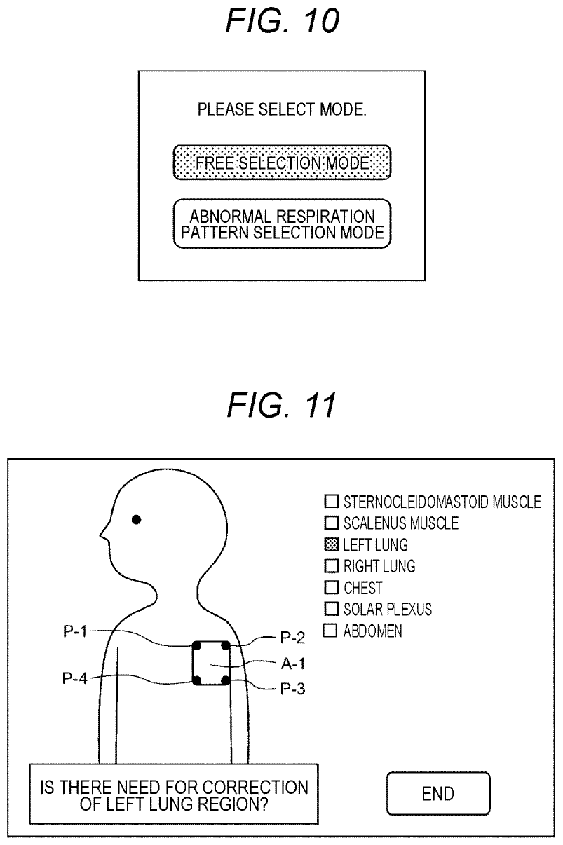

FIG. 9 is a flowchart for explaining a specific example of the procedure of designating the body region designation position, and a specific example of the procedure of designating the body region designation position will be described using the screen examples of the display unit of FIGS. 10 to 15 as appropriate.

In FIG. 9, in step S11, the user selects one of an abnormal respiration pattern mode in which the designation of the body region position of interest with respect to the patient image from the camera 11 displayed on the display unit is selected from the type of abnormal respiration and a free selection mode in which the designation is freely selected. FIG. 10 is a selection screen for selecting either the abnormal respiration pattern mode or the free selection mode. The user selects and determines a button indicating a mode to be selected on the selection screen of FIG. 10. Here, it is assumed that the user selects the free selection mode. In this case, in FIG. 9, the process proceeds from step S11 to step S12.

In step S12, the screen of FIG. 11 is displayed on the display unit, and the user checks the checkbox corresponding to the body region to be observed. As a result, the candidate region A-1 corresponding to the body region in which the checkbox is checked is displayed to be superimposed on the patient image displayed on the display unit as a candidate for the body region designation position. The candidate region A-1 is set on the basis of a predetermined standard relative positional relationship with respect to the body region as described with reference to FIG. 5. FIG. 11 illustrates a case where the user checks the checkbox of the left lung, and a quadrangular candidate region A-1 that is superimposed on the patient image and has vertices P-1 to P-4 is displayed. In a case where the user needs to correct the candidate region A-1 on the screen of FIG. 11, the process proceeds to step S13, and in a case where the user does not need to correct the candidate region A, the process proceeds to step S14.

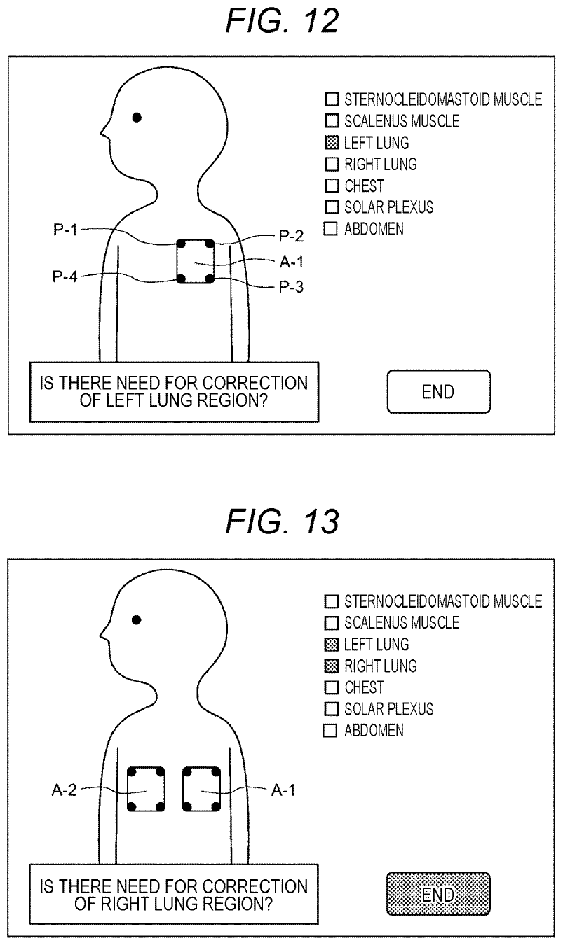

In step S13, the user selects (designates) the position of the first point as the body region designation position with reference to the candidate region A-1 displayed on the screen of FIG. 11. In a case where a point is designated as the body region, the designated position is designated as the body region designation position. In a case of designating the region as the body region, the user designates the second and subsequent positions on condition that three or more positions are designated. In a case where only one point is designated by the user, the body region designation unit 51 sets the position as the body region designation position. In a case where three or more positions are designated by the user, the body region designation unit 51 sets the region having vertices at the designated positions as the body region designation positions. When the correction of the candidate region A-1 is completed, the process proceeds from step S13 to step S14. FIG. 12 illustrates the screen of the display unit after the correction of the candidate region A-1 is performed by the user in step S13.

In step S14, in a case where the user wants to additionally designate the second body region, the user newly checks a checkbox corresponding to the body region to be added. FIG. 13 illustrates a screen of the display unit in a case where a second body region is additionally designated. The example of FIG. 13 illustrates a case where the user newly checks the checkbox of the right lung, and the candidate region A-2 is additionally displayed to be superimposed on the patient image. In a case where the user needs to correct the candidate region A-2 on the screen of FIG. 13, the user corrects the candidate region A-2 in a manner similar to step S13. In a case of ending the designation of the body region, the user selects the end button at the bottom of the screen.

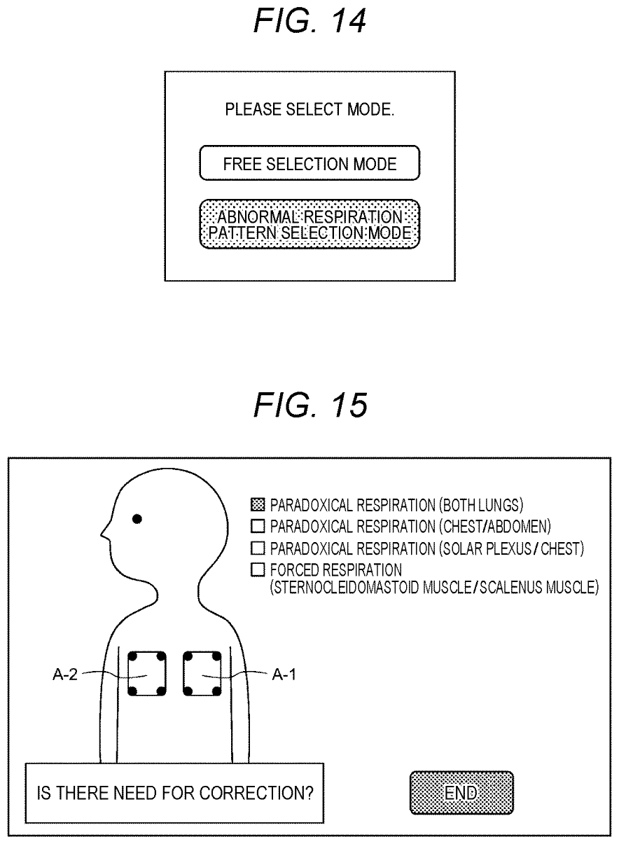

In step S11, when the user selects the button labeled as the abnormal respiration pattern selection mode as in the selection screen in FIG. 14, the process proceeds from step S11 to step S15. In step S15, the user checks a checkbox corresponding to the type of abnormal respiration desired to be observed. FIG. 15 illustrates a screen displayed on the display unit in a case where the abnormal respiration pattern selection mode is selected. In the example of FIG. 15, as the type of abnormal respiration, paradoxical respiration (both lungs), paradoxical respiration (chest/abdomen), paradoxical respiration (solar plexus/chest), and forced respiration (sternocleidomastoid muscle/scalenus muscle) can be selected. Paradoxical respiration (both lungs) is selected in a case where both the left and right lungs are designated as body region designation positions to detect abnormality of respiration. Paradoxical respiration (chest/abdomen) is selected in a case where the chest and the abdomen are designated as body region designation positions and abnormality of respiration is detected. Paradoxical respiration (solar plexus/chest) is selected in a case where the solar plexus and the chest are designated as body region designation positions and abnormality of respiration is detected. The forced respiration (sternocleidomastoid muscle and scalenus muscle) is selected in a case where the sternocleidomastoid muscle and the scalenus muscle are designated as the body region designation position and the abnormality of the respiration is detected. In the example of FIG. 15, the checkbox corresponding to paradoxical respiration (both lungs) is checked, and candidate regions A-1 and A-2 are displayed to be superimposed on the positions of the left and right lungs of the patient image. On the screen of FIG. 15, in a case where the user needs to correct the candidate region A-1 or A-2, the process proceeds to step S13 described above, and the user corrects the candidate region A-1 or A-2. In a case where the user needs to correct the candidate region A-1 or A-2, the process proceeds to step S16, and the user selects the end button displayed at the bottom of the screen of FIG. 15. As a result, the designation of the body region is completed.

### <Configuration Example of Computer>

The series of processing of the information processing apparatus 12 described above can be executed by hardware or software. In a case where a series of processing is executed by software, a program included in the software is installed on a computer. Here, examples of the computer include a computer incorporated in dedicated hardware, and for example, a general-purpose personal computer that can execute various functions by installing various programs.

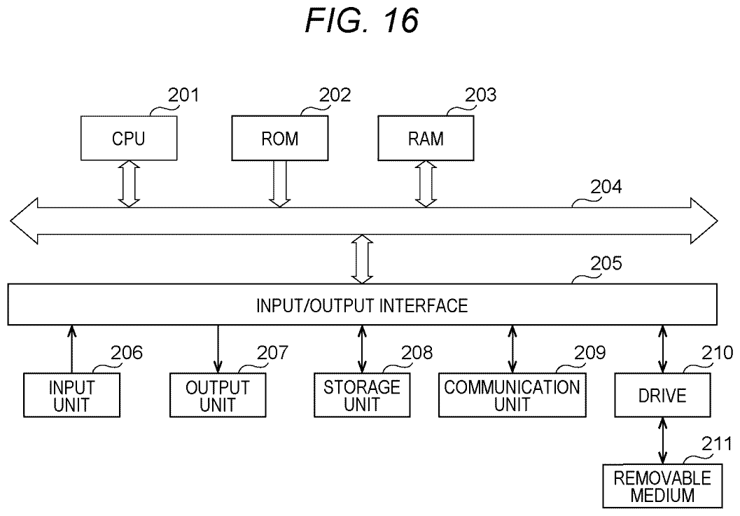

FIG. 16 is a block diagram illustrating a configuration example of hardware of a computer that executes the above-described series of processing by a program.

In the computer, a central processing unit (CPU) 201, a read only memory (ROM) 202, and a random access memory (RAN) 203 are mutually connected by a bus 204.

An input/output interface 205 is further connected to the bus 204. The input/output interface 205 is connected to an input unit 206, an output unit 207, a storage unit 208, a communication unit 209, and a drive 210.

The input unit 206 includes a keyboard, a mouse, a microphone, and the like. The output unit 207 includes a display, a speaker, and the like. The storage unit 208 includes a hard disk, a nonvolatile memory and the like. The communication unit 209 includes a network interface, and the like. The drive 210 drives a removable medium 211 such as a magnetic disk, an optical disk, a magneto-optical disk, or a semiconductor memory.

In the computer configured as described above, for example, the CPU 201 loads the program stored in the storage unit 208 into the RAM 203 via the input/output interface 205 and the bus 204 and executes the program, thereby performing the above-described series of processing.

The program executed by the computer (CPU 201) can be provided by being recorded on the removable medium 211 as a package medium or the like, for example. Further, the program can be provided via a wired or wireless transmission medium such as a local area network, the Internet, or digital satellite broadcasting.

In the computer, the program can be installed in the storage unit 208 via the input/output interface 205 by mounting the removable medium 211 in the drive 210. Furthermore, the program can be received by the communication unit 209 through the wired or wireless transmission medium to be installed on the storage unit 208. Additionally, the program may be installed in advance on the ROM 202 and the storage unit 208.

Note that the program executed by the computer may be a program in which processing is performed in time series in the order described in the present specification or may be a program in which processing is performed in parallel or at necessary timing such as when a call is made.

Here, in the present specification, the process to be performed by the computer in accordance with the program is not necessarily performed in time series according to orders described in the flowcharts. That is, the processing to be performed by the computer in accordance with the program includes processing to be executed in parallel or independently of one another (parallel processing or object-based processing, for example).

Furthermore, the program may be processed by one computer (one processor) or processed in a distributed manner by a plurality of computers. Moreover, the program may be transferred to a distant computer to be executed.

Moreover, in the present specification, a system means a set of a plurality of components (apparatuses, modules (parts), and the like), and it does not matter whether or not all the components are in the same housing. Therefore, a plurality of apparatuses housed in separate housings and connected to each other via a network and one apparatus in which a plurality of modules is housed in one housing are both systems.

Furthermore, for example, a configuration described as one apparatus (or one processing unit) may be divided and configured as the plurality of the apparatuses (or the processing units). Conversely, configurations described above as a plurality of apparatuses (or processing units) may be collectively configured as one apparatus (or processing unit). Furthermore, it goes without saying that a configuration other than the above-described configurations may be added to the configuration of each apparatus (or each processing unit). Moreover, when the configuration and operation as the entire system are substantially the same, a part of the configuration of a certain apparatus (or processing unit) may be included in the configuration of another apparatus (or another processing unit).

Furthermore, for example, the present technology can be configured as cloud computing in which one function is shared and jointly processed by the plurality of the apparatuses through the network.

Furthermore, for example, the program described above can be executed by any apparatus. In this case, the apparatus is only required to have a necessary function (functional block and the like) and obtain necessary information.

Furthermore, for example, each step described in the flowcharts described above can be executed by one apparatus, or can be executed in a shared manner by the plurality of the apparatuses. Moreover, in a case where a plurality of processes is included in one step, the plurality of the processes included in the one step can be executed by one apparatus or shared and executed by the plurality of the apparatuses. In other words, the plurality of the processes included in one step can also be executed as processes of a plurality of steps. Conversely, the processes described as the plurality of the steps can also be collectively executed as one step.

Note that, in the program to be executed by the computer, the processes in steps describing the program may be executed in time series in the order described in the present specification, or may be executed in parallel, or independently at a necessary timing such as when a call is made. That is, unless there is a contradiction, the process in the each step may also be executed in an order different from the orders described above. Moreover, the processes in the steps describing the program may be executed in parallel with processes of another program, or may be executed in combination with processes of the other program.

Note that, a plurality of the present technologies that has been described in the present specification can each be implemented independently as a single unit unless there is a contradiction. Of course, a plurality of arbitrary present technologies can be implemented in combination. For example, a part or all of the present technologies described in any of the embodiments can be implemented in combination with a part or all of the present technologies described in other embodiments. Furthermore, a part or all of any of the above-described present technologies can be implemented together with another technology that is not described above.

### <Example of Combination of Configurations>

Note that the present technology can also have the following configurations.

(1)

An information processing system including:
an estimation unit that estimates positions of joint points of a target person from an image of the target person captured for each frame;
a calculation unit that calculates a relative positional relationship between a position of a body region of the target person in the image of a first frame designated by a user and the joint points estimated by the estimation unit for the image of the first frame;
a decision unit that determines the position of the body region of the target person in the image of an arbitrary frame different from the first frame on the basis of the positions of the joint points estimated by the estimation unit for the image of the arbitrary frame and the relative positional relationship; and
a detection unit that detects a three-dimensional position change of the body region of the target person on the basis of the position of the body region of the target person in the image of the arbitrary frame.
   (2)

The information processing system according to (1), further including
a determination unit that determines abnormal respiration on the basis of the position change of the body region of the target person detected by the detection unit.
   (3)

The information processing system according to (2),
in which the body region of the target person is a chest and an abdomen, and
the determination unit makes a determination of the abnormal respiration on the basis of the position change of the chest and the position change of the abdomen.
   (4)

The information processing system according to (2) or (3),
in which the determination unit does not make the determination on the basis of a posture of the target person detected on the basis of the positions of the joint points estimated by the estimation unit.
   (5)

The information processing system according to (4),
in which the determination unit does not make the determination in a case where the posture of the target person is a prone position or a lateral decubitus position.
   (6)

The information processing system according to any one of (1) to (5), further including
a designation unit by which the user designates the body region of the target person,
in which the designation unit automatically designates the position of the body region corresponding to a type of the body region designated by the user.
   (7)

The information processing system according to (6),
in which the designation unit corrects the position designated of the body region on the basis of an operation by the user.
   (8)

The information processing system according to any one of (1) to (7),
in which the calculation unit calculates angle information and ratio information as the relative positional relationship.
   (9)

The information processing system according to (8),
in which the calculation unit calculates, as the angle information, an angle between a line segment connecting two of the joint points and a line segment connecting a position of each of the two of the joint points and the position of the body region designated by the user.
   (10)

The information processing system according to (9),
in which the calculation unit calculates the angle for each of a plurality of combinations in which one of the two of the joint points is different from other combinations, the plurality of combinations being combinations of the two of the joint points.
   (11)

The information processing system according to any one of (8) to (10),
in which the calculation unit calculates, as the ratio information, a ratio value at which a straight line passing through the position of the body region designated by the user divides each of a first line segment connecting two of the joint points and a second line segment different from the first line segment connecting the two of the joint points.
   (12)

The information processing system according to any one of (8) to (11),
in which the decision unit calculates the position of the body region by a weighted addition sum of the position of the body region determined on the basis of the angle information and the position of the body region determined on the basis of the ratio information.
   (13)

The information processing system according to any one of (1) to (12), further including
a designation unit that designates a type of abnormal respiration observed by the user,
in which the designation unit automatically designates the position of the body region corresponding to the type designated by the user.
   (14)

An information processing method in which
an information processing system includes
an estimation unit, a calculation unit, a decision unit, and a detection unit,
in which the estimation unit estimates positions of joint points of a target person from an image of the target person captured for each frame,
the calculation unit calculates a relative positional relationship between a position of a body region of the target person in the image of a first frame designated by a user and the joint points estimated by the estimation unit for the image of the first frame,
the decision unit determines the position of the body region of the target person in the image of an arbitrary frame different from the first frame on the basis of the positions of the joint points estimated by the estimation unit for the image of the arbitrary frame and the relative positional relationship, and
the detection unit detects a three-dimensional position change of the body region of the target person on the basis of the position of the body region of the target person in the image of the arbitrary frame.
   (15)

A program for causing a computer to function as:
an estimation unit that estimates positions of joint points of a target person from an image of the target person captured for each frame;
a calculation unit that calculates a relative positional relationship between a position of a body region of the target person in the image of a first frame designated by a user and the joint points estimated by the estimation unit for the image of the first frame;
a decision unit that determines the position of the body region of the target person in the image of an arbitrary frame different from the first frame on the basis of the positions of the joint points estimated by the estimation unit for the image of the arbitrary frame and the relative positional relationship; and
a detection unit that detects a three-dimensional position change of the body region of the target person on the basis of the position of the body region of the target person in the image of the arbitrary frame.

### REFERENCE SIGNS LIST

1 Information processing system
11 Camera
12 Information processing apparatus
13 In-hospital server
31 Body region setting unit
32 Body region tracking unit
51 Body region designation unit
52 Skeleton estimation unit
53 Relative position calculation unit
71 Body region position decision unit
72 Motion amount calculation unit
73 Abnormal respiration determination unit

1. An information processing system comprising:
an estimation unit that estimates positions of joint points of a target person from an image of the target person captured for each frame;
a calculation unit that calculates a relative positional relationship between a position of a body region of the target person in the image of a first frame designated by a user and the joint points estimated by the estimation unit for the image of the first frame;
a decision unit that determines the position of the body region of the target person in the image of an arbitrary frame different from the first frame on a basis of the positions of the joint points estimated by the estimation unit for the image of the arbitrary frame and the relative positional relationship; and
a detection unit that detects a three-dimensional position change of the body region of the target person on a basis of the position of the body region of the target person in the image of the arbitrary frame.

2. The information processing system according to claim 1, further comprising
a determination unit that determines abnormal respiration on a basis of the position change of the body region of the target person detected by the detection unit.

3. The information processing system according to claim 2,
wherein the body region of the target person is a chest and an abdomen, and
the determination unit makes a determination of the abnormal respiration on a basis of the position change of the chest and the position change of the abdomen.

4. The information processing system according to claim 2,
wherein the determination unit does not make the determination on a basis of a posture of the target person detected on a basis of the positions of the joint points estimated by the estimation unit.

5. The information processing system according to claim 4,
wherein the determination unit does not make the determination in a case where the posture of the target person is a prone position or a lateral decubitus position.

6. The information processing system according to claim 1, further comprising
a designation unit by which the user designates the body region of the target person,
wherein the designation unit automatically designates the position of the body region corresponding to a type of the body region designated by the user.

7. The information processing system according to claim 6,
wherein the designation unit corrects the position designated of the body region on a basis of an operation by the user.

8. The information processing system according to claim 1,
wherein the calculation unit calculates angle information and ratio information as the relative positional relationship.

9. The information processing system according to claim 8,
wherein the calculation unit calculates, as the angle information, an angle between a line segment connecting two of the joint points and a line segment connecting a position of each of the two of the joint points and the position of the body region designated by the user.

10. The information processing system according to claim 9,
wherein the calculation unit calculates the angle for each of a plurality of combinations in which one of the two of the joint points is different from other combinations, the plurality of combinations being combinations of the two of the joint points.

11. The information processing system according to claim 8,
wherein the calculation unit calculates, as the ratio information, a ratio value at which a straight line passing through the position of the body region designated by the user divides each of a first line segment connecting two of the joint points and a second line segment different from the first line segment connecting the two of the joint points.

12. The information processing system according to claim 8,
wherein the decision unit calculates the position of the body region by a weighted addition sum of the position of the body region determined on a basis of the angle information and the position of the body region determined on a basis of the ratio information.

13. The information processing system according to claim 1, further comprising
a designation unit that designates a type of abnormal respiration observed by the user,
wherein the designation unit automatically designates the position of the body region corresponding to the type designated by the user.

14. An information processing method in which
an information processing system includes
an estimation unit, a calculation unit, a decision unit, and a detection unit,
wherein the estimation unit estimates positions of joint points of a target person from an image of the target person captured for each frame,
the calculation unit calculates a relative positional relationship between a position of a body region of the target person in the image of a first frame designated by a user and the joint points estimated by the estimation unit for the image of the first frame,
the decision unit determines the position of the body region of the target person in the image of an arbitrary frame different from the first frame on a basis of the positions of the joint points estimated by the estimation unit for the image of the arbitrary frame and the relative positional relationship, and
the detection unit detects a three-dimensional position change of the body region of the target person on a basis of the position of the body region of the target person in the image of the arbitrary frame.

15. A program for causing a computer to function as:
an estimation unit that estimates positions of joint points of a target person from an image of the target person captured for each frame;
a calculation unit that calculates a relative positional relationship between a position of a body region of the target person in the image of a first frame designated by a user and the joint points estimated by the estimation unit for the image of the first frame;
a decision unit that determines the position of the body region of the target person in the image of an arbitrary frame different from the first frame on a basis of the positions of the joint points estimated by the estimation unit for the image of the arbitrary frame and the relative positional relationship; and
a detection unit that detects a three-dimensional position change of the body region of the target person on a basis of the position of the body region of the target person in the image of the arbitrary frame.
